(19) United States
(12) Patent Application Publication
(10) Pub. No.: US 2019/0125800 A1
(43) Pub. Date: May 2, 2019

(54) TYPE 2 INNATE LYMPHOID CELLS AND METHODS OF ENRICHING AND GROWING

(71) Applicant: Regents of the University of Minnesota, Minneapolis, MN (US)

(72) Inventors: Yan Xing, North St. Paul, MN (US); Heather Stefanski, Mendota Heights, MN (US); Bruce Blazar, Golden Valley, MN (US)

(21) Appl. No.: 16/176,912

(22) Filed: Oct. 31, 2018

Related U.S. Application Data
(60) Provisional application No. 62/580,072, filed on Nov. 1, 2017.

(51) Int. Cl. A61K 35/17; C12N 5/00; C12N 5/078

(52) U.S. Cl. CPC A61K 35/17; C12N 5/0018; C12N 2501/2302; C12N 2501/2325; C12N 2501/2333; C12N 5/0634

(57) ABSTRACT

Described herein are methods of enriching and growing Type 2 innate lymphoid cells.

### CROSS REFERENCE TO RELATED APPLICATIONS

This application claims the benefit of U.S. Ser. No. 62/580,072 filed Nov. 1, 2017, titled “Type 2 Innate Lymphoid Cells and Methods of Enriching and Growing,” and is incorporated herein by reference in its entirety.

### STATEMENT OF GOVERNMENT RIGHTS

This invention was made with government support under CA72669 awarded by the National Institutes of Health. The government has certain rights in the invention.

### BACKGROUND

Innate lymphoid cells (ILCs) are a family of immune cells known to play a role in immunity and inflammation and produce an array of secreted proteins, termed cytokines that direct the developing immune response. They appear distinct from other known immune cells though they mirror the phenotypes and functions of T cells secreting a similar suite of inflammatory mediators as T lymphocytes. However, in contrast to T cells, ILCs do not express antigen receptors or undergo clonal selection and expansion when stimulated.

Type 2 innate lymphoid cells (ILC2) have become an attractive therapeutic target for preventing and treating disease such as graft versus host disease, and for promoting tissue repair. However, pre-clinical research and clinical applications of ILC2 has been hampered by the limited cell numbers, for example low frequency (0.01-0.03%) in peripheral blood and a lack of an effective method to enrich or select and grow.

### SUMMARY

Described herein are methods for enriching and growing type 2 innate lymphoid cells.

In one aspect the invention includes, a method of growing Type 2 innate lymphoid (ILC2) cells, the method comprising:

culturing the ILC2 cells in culture media, the culture media comprising interleukin (IL-2), interleukin 25 (IL-25) and interleukin 33 (IL-33).

In another aspect of the invention is a method of growing ILC2 cells, the method comprising: enriching for ILC2; and culturing the ILC2 in culture media, the culture media comprising IL-2, IL-25 and IL-33.

In yet another aspect is a cell culture media comprising ILC2 cells, and IL-2, IL-25, and IL-33.

The resultant ILC2 is suitable for use in pre-clinical or clinical applications.

### BRIEF DESCRIPTION OF THE DRAWINGS

For the purpose of illustrating the invention, there are depicted in the drawings certain embodiments of the invention. However, the invention is not limited the precise arrangements and instrumentalities of the embodiments depicted in the drawings.

FIG. 1. A massive ex vivo expansion of human ILC2 cells. Human ILC2 cells were enriched from an apheresis unit of peripheral blood cells using RosetteSep human ILC2 enrichment kit, and cultured in α-MEM media/20% FBS with 20 IU/mL IL-2, 50 ng/mL IL-25 and 50 ng/mL IL-33 for 13 days. (A) A graph showing absolute cell numbers of CD161⁺ cells at each time point. Numbers in red denote fold change of ILC2 cell expansion. (B) A contour plot of flow cytometry showing expression of CD161 and CD294 on the surface of the cultured cells. (C) A histogram overlay of flow cytometry showing GATA3 expression following by intracellular staining (black thick line). Grey filled area represents negative control. (D) Cultured cells were stimulated by cell stimulation cocktail with protein inhibitors, and intracellular stained with IFNγ and IL-13 antibodies.

FIG. 2. A stable method to enrich human ILC2 cells. A graph showing the absolute cell numbers of Human ILC2 cells enriched from an apheresis unit of peripheral blood cells using RosetteSep human ILC2 enrichment kit. Data are representative of six separate experiments. Blood samples were from six donors. Each symbol represents an individual donor; small horizontal lines indicate the group mean (±s.d.).

FIG. 3. A purity and recovery comparison of human ILC2 cell enrichment methods. Human ILC2 cells were enriched from an apheresis unit of peripheral blood cells using RosetteSep human ILC2 enrichment kit, EasySep human ILC2 enrichment kit or a combination of EasySep human NK cell enrichment kit and EasySep human CD56 positive selection II kit. (A) Flow cytometric profiles of pre-enrichment and post-enrichment samples divided by linage makers (Lin) and CD127 in upper panels. Lower panels showing Lin⁻CD127⁺ gated populations divided by CD161 and CRTH2. Numbers in red denote percentages of ILC2 in live cells. (B, C) Graphs showing ILC2 cell percentage and enrichment yields of the indicated three kinds of enrichment methods.

FIG. 4. RosetteSep ILC2 kit enriched cell grow with the best proliferation rate. Enriched human ILC2 cells were cultured in α-MEM media/20% FBS with 20 IU/mL of IL-2, 50 ng/mL of IL-25, IL-33 and TSLP for 10 days. (A) Flow cytometric profiles of 10 days cultured human ILC2 cells. Cells were stimulated by cell stimulation cocktail with protein inhibitors, and followed by intracellular staining. (B) A graph showing cell numbers of IL-13-producing human ILC2 cells after 10-day culture. Numbers in red denote fold change of ILC2 cell expansion.

FIG. 5. IMDM media with high amount cytokines does not support ILC2 cell growth well. Enriched human ILC2 cells were cultured in IMEM media/10% FBS with 500 ng/mL of IL-2, IL-25, IL-33 and TSLP (IMDM500) for 10 days. (A) Flow cytometric profiles of 10-day cultured human ILC2 cells. Cells were stimulated by cell stimulation cocktail with protein inhibitors, and followed by intracellular staining. (B) A graph showing IL-13 single producing human ILC2 cells after 10 days culture. Numbers in red denote fold change of ILC2 cell expansion.

FIG. 6. OP9-DL1 co-culture did not enhance ILC2 production. Enriched human ILC2 cells were cultured in α-MEM media/20% FBS with 20 IU/mL of IL-2, 50 ng/mL of IL-25, IL-33 and TSLP with or without 10,000 cGy irradiated OP9-DL1 feeder cells for 10 days. A graph showing ILC2 cell numbers after 10-day culture.

FIG. 7. TSLP did not enhance ILC2 production. Enriched human ILC2 cells were cultured in α-MEM media/20% FBS with 20 IU/mL of IL-2, 50 ng/mL of IL-25 and IL-33 with or without TSLP for 10 days. (A) A graph showing ILC2 cell numbers with or without TSLP in culture after 10 days. (B) Flow cytometric profiles of 10-day cultured human ILC2 cells divided by expression of CD161 and CRTH2.

FIG. 8. IL-2 is required for ILC2 expansion. Following human ILC2 enrichments using RosetteSep kit, human ILC2 cells were sorted by FACSAria II based on the characters Lin⁻CD127⁺CD161⁺CRTH2⁺ of ILC2 cells, and then cultured in α-MEM media/20% FBS with 50 ng/mL of IL-25 and IL-33, with or without 50 ng/mL of TSLP or 20 IU/mL of IL-2 for 10 days. (A) A graph showing cell numbers of ILC2 cells after 10-day culture. (B) Flow cytometric profiles of 10-day cultured human ILC2 cells divided by expression of CD161 and CRTH2. (C) Cells were stimulated by cell stimulation cocktail with protein inhibitors for 4 hours, and then intracellularly stained using IL-5 and IL-13 antibodies.

### DETAILED DESCRIPTION

Although the present disclosure provides references to embodiments, persons skilled in the art will recognize that changes may be made in form and detail without departing from the spirit and scope of the invention. Reference to various embodiments does not limit the scope of the claims attached hereto. Additionally, any examples set forth in this specification are not intended to be limiting and merely set forth some of the many possible embodiments for the appended claims. Preferred methods and materials are described below, although methods and materials similar or equivalent to those described herein can be used in practice or testing of the present disclosure. All publications, patent applications, patents and other references mentioned herein are incorporated by reference in their entirety.

### Definitions

Unless otherwise defined, all technical and scientific terms used herein have the same meaning as commonly understood by one of ordinary skill in the art. In case of conflict, the present document and its definitions will control.

The terms “cell culture medium,” “culture medium” (plural “media” in each case), and “media,” refer to a nutritive solution for culturing cells.

The term “culturing” refers to the growth or maintenance of cells in an artificial, environment in vitro.

The term “cytokine” refers to a compound that induces a physiological response in a cell, such as growth, differentiation, senescence, apoptosis, cytotoxicity or antibody secretion. Included in this definition of ‘cytokine’ are growth factors, interleukins, colony-stimulating factors, interferons and lymphokines.

The term “enriching” refers to a cell or cells, which are chosen or selected over other cells and which are isolated from other cells such that these cells are not associated with one or more cells or one or more cellular components that are associated with the cell or cells in vivo.

The term “growing,” “growth,” or grammatical equivalents thereof when used in context of cells refers to the increase in cell number, cell size, or both.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The term “optional” or “optionally” means that the subsequently described event or circumstance may but need not occur, and that the description includes instances where the event or circumstance occurs and instances in which it does not.

The term “about” modifying, for example, the quantity of an ingredient in a composition, concentration, volume, temperature, time, yield, flow rate, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods, and like proximate considerations. Where “about” is employed to describe a range of values, for example “about 1 to 5” the recitation means “1 to 5” and “about 1 to about 5” and “1 to about 5” and “about 1 to 5” unless specifically limited by context.

The term “substantially” means “consisting essentially of”, as that term is construed in U.S. patent law, and includes as a subset thereof “consisting of” as that term is construed in U.S. patent law. For example, a solution that is “substantially free” of a specified compound or material may be free of that compound or material, or may have a minor amount of that compound or material present, such as through unintended contamination, side reactions, or incomplete purification. A “minor amount” may be a trace, an unmeasurable amount, an amount that does not interfere with a value or property, or some other amount as provided in context. A composition that has “substantially only” a provided list of components may consist of only those components, or have a trace amount of some other component present, or have one or more additional components that do not materially affect the properties of the composition. Additionally, “substantially” modifying, for example, the type or quantity of an ingredient in a composition, a property, a measurable quantity, a method, a value, or a range, employed in describing the embodiments of the disclosure, refers to a variation that does not affect the overall recited composition, property, quantity, method, value, or range thereof in a manner that negates an intended composition, property, quantity, method, value, or range. Where modified by the term “substantially” the claims appended hereto include equivalents according to this definition.

As used herein, any recited ranges of values contemplate all values within the range and are to be construed as support for claims reciting any sub-ranges having endpoints which are real number values within the recited range. For example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5 and any values in between.

### DESCRIPTION

Described herein are methods of isolating and enriching Type 2 innate lymphoid (ILC2) cells. In embodiments, methods are provided for isolating, expanding, activating and identifying ILC2 cells, in some embodiments in vitro and/or ex vivo. In embodiments, the enriched ILC2 are used in pre-clinical, clinical, and therapeutic methods and compositions.

### Enriching for ILC2

In embodiments, the ILC2 are selected or enriched. In embodiments, the enrichment or selection is via negative selection. Various commercially available kits are used to isolate and enrich for ILC2. In embodiments, the commercially available kits are RosetteSep™ kits (e.g., human ILC2 enrichment), EasySep™ kits (e.g., human ILC2 enrichment kit), and EasySep™ kits (e.g., human NK-cell-negative selection and EasySep™ human CD56-positive selection II kit). In embodiments, the kits used are RosetteSep™ ILC2 kit, EasySep™ ILC2 and EasySep™ NK+CD56 or combinations thereof. In embodiments, the kit used is RosetteSep™ Human. ILC2 enrichment kit. Using the manufacturer's instructions, the commercial kits can be used to isolate and enrich for ILC2.

In embodiments, the ILC2 cells are isolated directly from blood, tissue obtained via biopsy, autopsy, donation or other surgical or medical procedure. In embodiments, the ILC2 cells are isolated from peripheral blood, stem cells, bone marrow or combinations thereof. In embodiments, the peripheral blood is from human including for example mammalian blood. In other embodiments, ILC2 cells can be isolated and enriched from blood products. In some embodiments, the ILC2 cells can be derived from human cord blood cells. Such human cord blood cells can be derived from a donor subject and/or from a patient's own cord blood.

The amount of ILC2 cells initially obtained and used for further selection range from 0.001% to 0.16% of total cells, or from 0.02% to about 0.8% of total cells.

In embodiments, the percentage of ILC2 cells enriched range from 0.5 to 15%, from 1.0 to 10% or from 1.0 to 7% compared to the initially obtained ILC2 cells of total cells.

In embodiments, the enriched cells result in yield from 1.0×10⁴ to 5.0×10⁶; from 4.0×10⁴ to 4×10⁶ or from 1.0×10⁴ to 4.0×10⁵ to. In embodiments, the ILC2 cells are enriched greater than 10 to 100 fold.

### Culturing of ILC2

In embodiments, ILC2 cells used for culturing are obtained commercially, for example from ATCC (Rockville, Md.), Cell Systems, Inc. (Kirkland, Wash.) or Invitrogen Corporation (San Diego, Calif.) or isolated from the appropriate samples. In embodiments, ILC2 cells are isolated from peripheral blood. In other embodiments, the ILC2 cells are obtained from human peripheral blood. Whatever the method of enriching or selecting, ILC2 cells are cultured to grow the cell numbers, cell size, or both in vitro. In an embodiment, the method includes culturing ILC2 cells with a culture medium; the culture medium comprises, consists essentially of, or consists of IL-2, IL-33, and IL-25, referred herein as cytokine cocktail. In embodiments, the culturing is from about 3 to 30 days or 5 to 20 days or from 5 to 10 days. In an embodiment, culturing is for 10 days.

In embodiments, IL-25 and IL-33 are present in the culture media from about 20-100 ng/mL, 30-100 ng/mL, from 30-50 ng/mL or from 40-60 ng/mL.

In embodiments, IL-2 is present from 10 IU/mL to 50 IU/mL. In embodiments, the IL-25 and IL-33 are present in the culture media at 50 ng/mL with IL-2 is present at 20 IU/mL. In an embodiment the ILC2 cell growth is greater than 10 to 100 fold. In embodiments, the ILC2 cell growth is greater than 1000 fold.

In embodiments, the ILC2 cell growth is greater than 1000 fold relative to enriching ILC2 using EasySep™ ILC2 kit. In other embodiments the ILC2 cell growth is greater than 10 fold relative enriching ILC2 using MAC beads.

In one embodiment, the cytokine cocktail may be present in a “basal media.” Any basal medium may be used in accordance with the disclosure. Examples of basal media include but are not limited to Minimum Essential Media (MEM), Dulbecco's Modified Eagle Medium (DMEM), Dulbecco's Modified Eagle's Medium/Nutrient Mixture F-12 (DMEM/F12), RPMI-1640, Basal Medium Eagle (BME), and Iscove's Modified Dulbecco's Medium (IMDM). In an embodiment, the basal media is α-MEM. Ingredients which the basal media of the present disclosure may include are amino acids, lipids, fatty acids, hydrolysates from non-animal sources, vitamins, organic and/or inorganic salts, trace elements, buffering salts and sugars, and modified derivatives of such ingredients.

In embodiments, the cell density used for culturing the cells is from 1×10⁵/mL to 5×10⁶/mL.

In embodiments, the resultant ILC2 cells are greater than 90% pure; from 90-100% pure; from 90-99% pure; or from 95% to 100% pure.

In embodiments, the resultant ILC2 cells express CD161 and GATA. In embodiments, the cells produce IL-13 and IL-5 in culture when exposed to phorbol myristate acetate (PMA), ionomycin or both, but do not produce IFN-gamma. In embodiments, the resultant ILC2 cells express CD161 and GATA and express IL-13 and IL-5.

The disclosed methods can provide ILC2 cells for use in pharmaceutical compositions and related therapeutic methods and approaches.

Additional embodiments include:

1. A method of growing Type 2 innate lymphoid (ILC2) cells, the method comprising:

culturing the ILC2 cells in culture media, the culture media comprising interleukin (IL-2), interleukin 25 (IL-25) and interleukin 33 (IL-33).

2. The method of embodiment 1, further comprises enriching for ILC2.

3. A method of growing ILC2 cells, the method comprising:

enriching for ILC2; and

culturing the ILC2 in culture media, the culture media comprising IL-2, IL-25 and IL-33.

4. The method of anyone of embodiments 1 to 3, wherein culturing is for about 3 to about 30 days.

5. The method of anyone of embodiments 1 to 3, wherein the culturing is for 10 days.

6. The method anyone of embodiments 1 to 5, wherein the IL-25 and IL-33 each comprises a concentration of about 30 ng/mL to 100 ng/mL and the IL-2 comprises 10 IU/mL to 50 IU/mL.

7. The method anyone of embodiments 1 to 5, wherein the, IL-25 and IL-33 each comprises a concentration of about 50 ng/mL and the IL-2 comprises 20 IU/mL.

8. The method of anyone of embodiments 1 to 7, wherein the culture media further comprises basal media.

9. The method of anyone of embodiments 1 to 8, wherein the culture media is free or substantially free of thymic stromal lymphopoietin (TSLP).

10. The method of anyone of embodiments 1 to 9, wherein the culture media is free or substantially free of OP9-DL1.

11. The method of anyone of embodiments 1 to 10, wherein the culture media consists essentially of IL-2, IL-25, and IL-33.

12. The method of anyone of embodiments 1 to 8, wherein the culture media consists of IL-2, IL-25, and IL-33.

13. The method of anyone of embodiments 2 to 12, wherein of enriching ILC2 cells are from blood.

14. The method of anyone of embodiments 2 to 13, wherein of enriching ILC2 cells are from peripheral blood.

15. The method of anyone of embodiments 2 to 14 wherein of enriching ILC2 cells are from human blood.

16. The method of anyone of embodiments 2 to 15, wherein of enriching ILC2 cells are by negative selection.

17. The method of anyone of embodiments 1 to 16, wherein the method results in an increase in the ILC2 cells by 100 fold relative to enriching ILC2 using EasySep™ ILC2 kit.

18. The method of anyone of embodiments 1 to 17, wherein the method results in an increase in the ILC2 cells by 10 fold relative enriching ILC2 using MAC beads.

19. The method of anyone of embodiments 1 to 18, wherein the ILC2 cells are from 90% to 100% pure.

20. The method of anyone of embodiments 1 to 19, wherein the method is in vitro or ex vivo.

21. The method of anyone of embodiments 1 to 20, further comprising analyzing the ILC2 for expression of CD161 and GATA3.

22. The method of anyone of embodiments 1 to 21, further comprising analyzing the ILC2 for production of interleukin IL-5 and IL-13.

23. A composition comprising ILC2 produced by the method of any one of embodiments 1 to 22.

24. ILC2 cells produced by the method of any one of embodiments 1 to 23.

25. A cell culture media comprising ILC2 cells, and IL-2, IL-25, and IL-33.

26. A cell culture media consisting essentially of ILC2 cells, IL-2, IL-25, and IL-33.

27. A cell culture media consisting of ILC2 cells, IL-2, IL-25 and IL-33.

28. A cell culture media of embodiment 25 comprising ILC2 cells, IL-2, IL-25, IL-33, and free from or substantially free from OP9-DL.

29. A cell culture media of anyone of embodiments 25 to 26 comprising ILC2 cells, IL-2, IL-25, IL-33, and free from or substantially free from TSLP.

30. A cell culture media of anyone of embodiments 25 to 27 comprising ILC2 cells, IL-2, IL-25, IL-33, and free from or substantially free from IL-7.

31. A cell culture media comprising IL-2, IL-25, and IL-33.

32. A cell culture media consisting essentially of IL-2, IL-25, and IL-33.

33. A cell culture media consisting of IL-2, IL-25, and IL-33.

34. The cell culture media of anyone of embodiments 25 to 33, wherein the IL-25 and IL-33 each comprises a concentration of about 30 ng/ml to 100 ng/ml and the IL-2 comprises 10 IU/mL to 50 IU/mL.

35. The cell culture media of anyone of embodiments 25 to 33, wherein the, IL-25 and IL-33 each comprises a concentration of about 50 ng/mL and the IL-2 comprises 20 IU/mL.

36. A cell culture media of any one of embodiments 31-33, wherein the culture media is IL-2, IL-free from or substantially free from OP9-DL.

37. A cell culture media of anyone of embodiments 31-33 wherein the culture media is IL-2, IL-25, IL-33, and free from or substantially free from TSLP.

38. A cell culture media of anyone of embodiments 31-33 is IL-2, IL-25, IL-33, and free from or substantially free from IL-7.

39. A kit comprising one or more containers of cell culture medium as in any one of embodiments 25 to 38, together with instructions for use.

40. Use of a cell culture medium as in any one of embodiments 1-39.

41. Use of ILC2 cells according to embodiment 40, for pre-clinical or clinical use.

### EXAMPLES

The invention is now described with reference to the following Examples. These examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compositions of the present invention and practice the claimed methods. The following working examples therefore, specifically point out the preferred embodiments of the present invention, and are not to be construed as limiting in any way the remainder of the disclosure.

### Example 1

Ex Vivo Expansion of Human ILC2 Cells

To determine if human ILC2 cells could be enriched and expanded ex vivo. An analysis of ILC2 enrichment from apheresis units of human peripheral blood cells were performed, and cell culture conditions for ILC2 expansion were investigated.

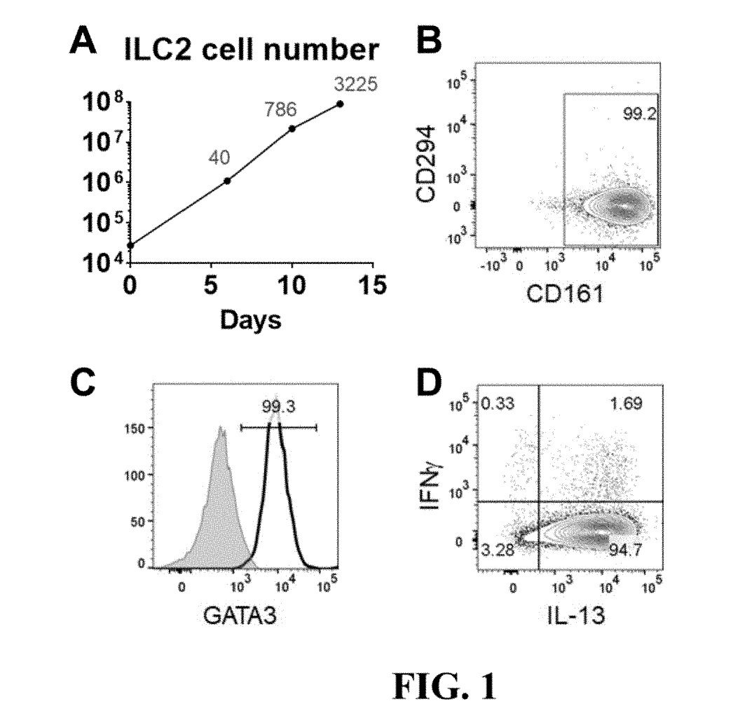

The figures show the results of those investigations. A combination of ILC2 cell enrichment method, culture media and cytokines ex vivo achieved human ILC2 cell expansion to thousands folds during around 13 days (FIG. 1A). After 13-day culture, more than 95% of the cells express CD161 and GATA3 (FIGS. 1B and 1C), but low level expression of CRTH2, which is consistent to CRTH2 down-regulation expression of ILC2 cell in culture previously reported by others. In addition, upon PMA and Ionomycin (Cell Stimulation Cocktail) stimulation for 4 hours, around 95% of the cultured cells produced IL-13, but not IFNγ (FIG. 1D). Therefore, the disclosed method allows ex vivo expansion of human ILC2 cells and maintains ILC2 major phenotypes and cytokine productive functions.

### Example 2

Human ILC2 Cell Enrichment Method

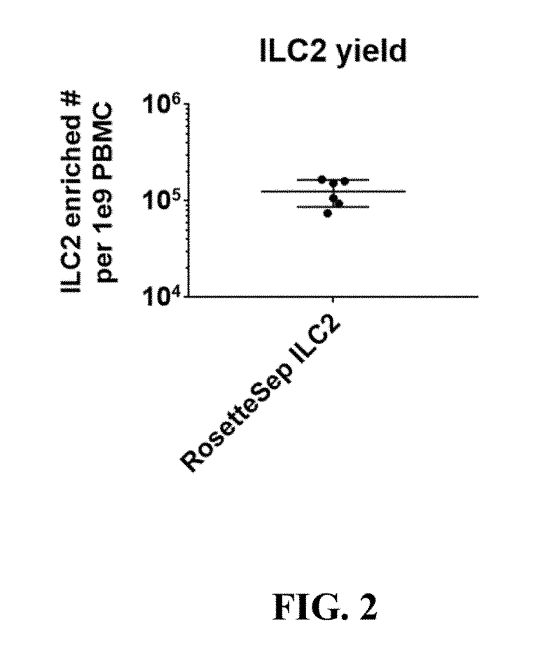

Human ILC2 cells are characterized by negative for linage markers (Lin⁻: CD1a⁻Cd11c⁻CD14⁻CD19⁻CD34⁻CD94⁻CD123⁻FceR1α⁻TCRαβ⁻TCRγδ⁻ and expression of the cytokine receptor subunit IL-7Rα (CD127), the natural killer cell receptor CD161 and the marker CRTH2 (CD294). Consistent with 0.01-0.03% of in the blood of normal healthy humans, ILC2 was identified at the range of 0.023-0.062% of total CD45⁺ cells in the apheresis units of peripheral blood cells. RosetteSep™ is a procedure for the isolation of untouched cells directly from whole blood. By crosslinking unwanted cells to red blood cells present in the sample, target cells are purified during standard density gradient centrifugation. Following RosetteSep™ Human ILC2 enrichment, the average yield of ILC2 cells from 10⁹ peripheral blood mononuclear cells (PBMC) is 10⁵ (FIG. 2).

### Example 3

Comparison of Human ILC2 Enrichment Methods for Purity and Recovery Rate

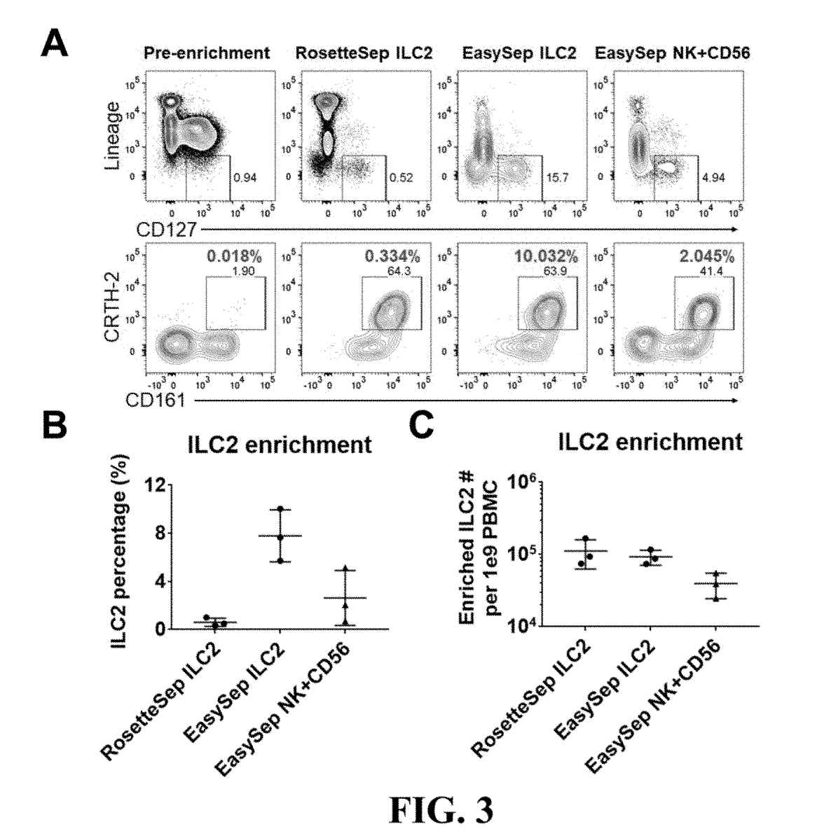

In addition to the RosetteSep™ ILC2 kit, the ILC2 cells were also enriched using an EasySep™ Human ILC2 enrichment, and a combination of EasySep™ NK-cell-negative selection and EasySep™ CD56-positive selection II kit (EasySep™ NK+CD56). The three different kits and method were compared for cell enrichment, purity and recovery rate. Among the three enrichment methods, EasySep™™ ILC2 kit and EasySep™™ NK+CD56 kits produced higher purity of ILC2 cells (8% and 5%, respectively) than RosetteSep™ ILC2 kit (FIG. 3A). However, RosetteSep™ kit and EasySep™™ ILC2 kit have higher recovery rate than EasySep™™ NK+CD56 kit (FIG. 3B).

### Example 4

Comparison Methods for ILC2 Cell Expansion

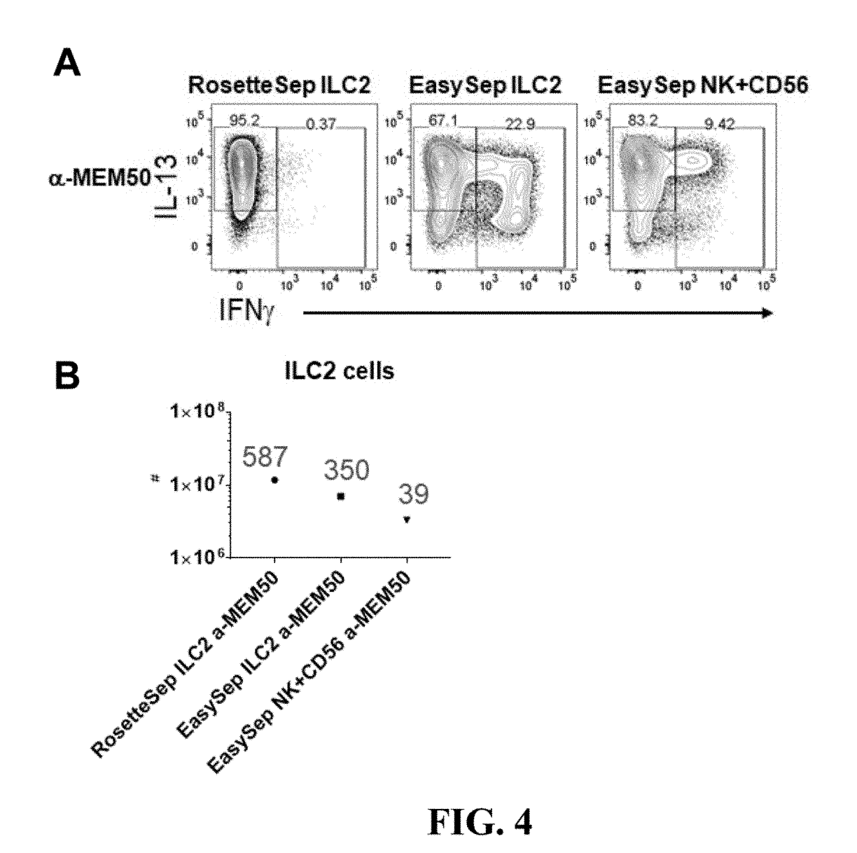

Following the human ILC2 enrichment, the cells were cultured in α-MEM media/20% FBS with 20 IU/mL of recombinant human IL-2, and 50 ng/mL of each of IL-25, IL-33 and TSLP (α-MEM50) for 10 days. After human ILC2 cells enriched by RosetteSep™ kit were cultured for 10 days, 95% of the cells produced IL-13 but not IFNγ upon PMA and lonomycin (Cell Stimulation Cocktail) stimulation, which is consistent to the characters of human ILC2 cells. In contrast, after cells enriched by EasySep™ ILC2 kit or EasySep™ NK+CD56 kits were cultured for 10 days, 10% to 25% of the cells produce IFNγ, which are characterized as ILC1-like cells (FIG. 4A). Thus, although the cells enriched by EasySep™ ILC2 kit and NK+CD56 kits include higher percentages of ILC2, more proportion of those cells preferentially became IFNγ-producing ILC1-like phenotype during culture. In contrast, nearly none of the cells enriched by RosetteSep™ kit became IFNγ-producing ILC1-like phenotype. In addition, human ILC2 cells enriched by RosetteSep™ kit expanded 587 folds after 10 days culture. The expansion rate is better than the cells enriched by EasySep™ ILC2 kit and EasySep™ NK+CD56 kits (FIG. 4B). Therefore, among the three ILC2 enrichment methods, RosetteSep™ human ILC2 enrichment kit is the best method to isolate ILC2 cells for ex vivo expansion.

### Example 5

IMDM Media with High Amount Cytokines does not Support ILC2 Cell Growth Well

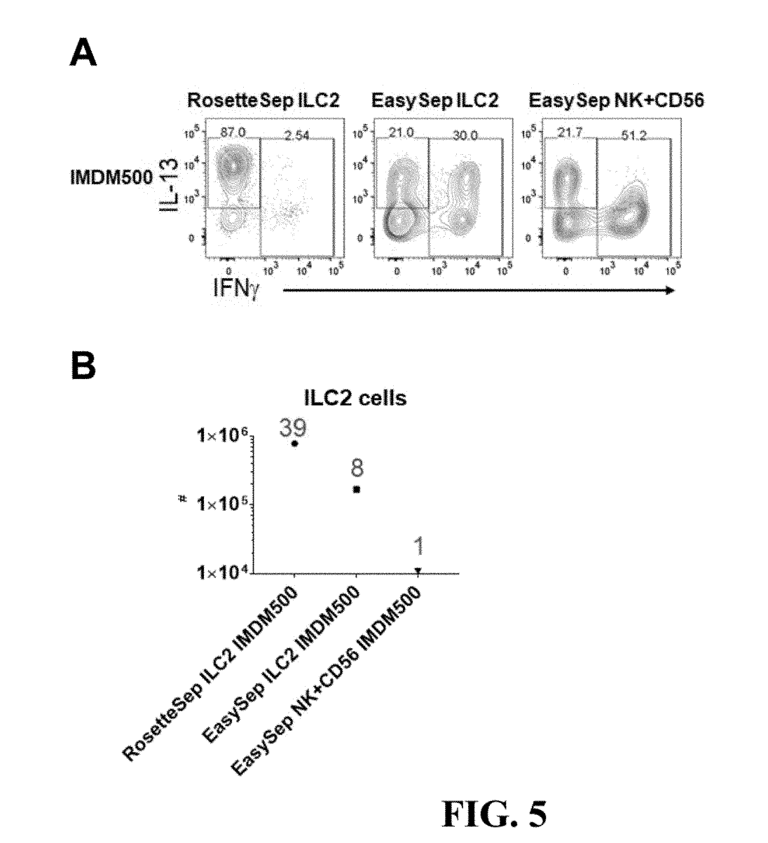

It was reported that ILC2 cells expanded 10.4-fold in IMDM medium/10% FBS with 500 ng/mL of each of recombinant human IL-2, IL-25, IL-13 and TSLP (IMDM500) for 5 days. Using this reported MDM500 medium, the human ILC2 cells enriched by the mentioned three enrichment methods (using the requisite kits), the IMDM500 condition was not found to expand ILC2 cells well. The IMDM500 gave rise to higher percentage of IFNγ-producing ILC1-like cells (FIG. 5).

### Example 6

OP9-DL1 Co-Culture Did not Enhance ILC2 Production

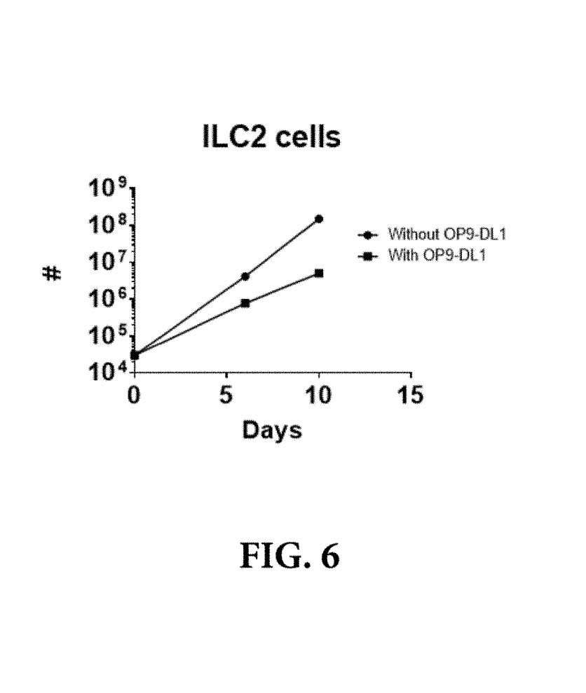

Recent studies reported that constitutive activation of Notch may induce prominent ILC2 differentiation. Given OP9-DL1 co-culture system provides Notch ligand Delta like 1 (DLL1) to activate Notch signaling, the OP9-DL1 co-culture system to ILC2 culture was used. OP9-DL1 cells are a bone-marrow-derived stromal cell line that ectopically expresses the Notch ligand, Delta-like 1 (Dll1). Compared to the culture without OP9-DL1 co-culture, ILC2 cells in OP9-DL1 co-culture did not increase in cell number for ILC2. On the contrary, ILC2 increase in cell numbers in OP9-DL1 co-culture was observed to be worse than without OP9-DL1 co-culture (FIG. 6).

### Example 7

TSLP does not Enhance ILC2 Production

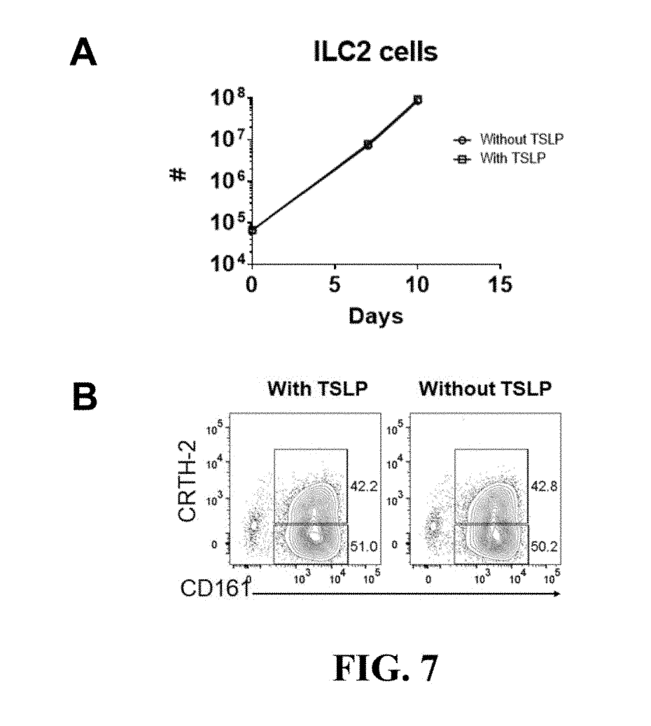

To further identify the key role of the cytokines for ILC2 expansion, we conducted comparison of various cytokine combinations. Thymic Stromal Lymphopoietin (TSLP) has recently been reported to play an essential role for IL-33- and IL-25-independent ILC2 cells in promoting skin inflammation. We tested if TSLP is critical for human circulating ILC2 expansion and how TSLP affects ILC2 phenotypes in vitro. The results showed that TSLP did not affect cell proliferation at all (FIG. 7A). Regardless of the presence or not of TSLP in cell culture, over 90% of the cells express CD161 (FIG. 7B). It suggests that TSLP is not critical for ILC2 expansion.

### Example 8

IL-2 is Required for ILC2 Cell Expansion.

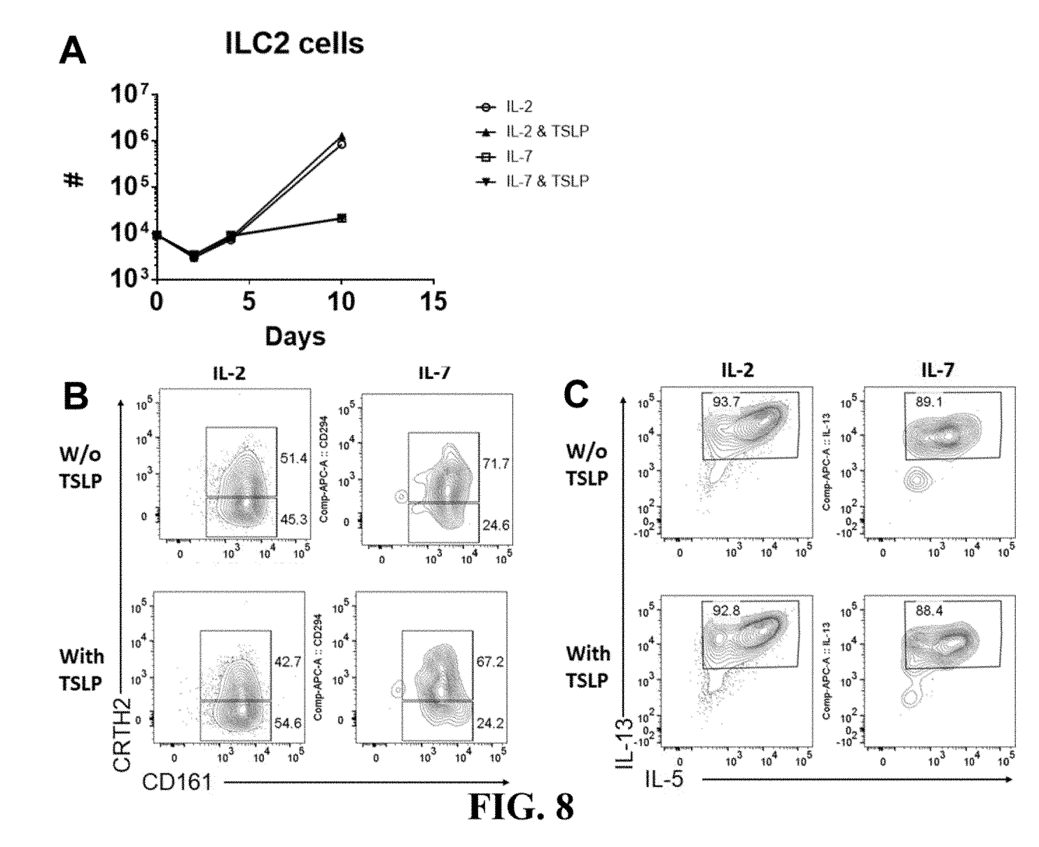

We further assessed the roles of IL-2 and IL-7 in ILC2 cell culture. Compared to 100-fold expansion of ILC2 cells in the presence of IL-2, cell numbers were almost not increased in IL-2-absence conditions even with the presence of IL-7 (FIG. 8A). Interestingly, In the IL-7 present conditions, ILC2 cells express higher CRTH2 level than that in absence of IL-7 (FIG. 8A), although the mechanism is not yet determined. Furthermore, we assessed the functions of cytokine production of the cultured ILC2 cells. Most of the cells (about 90% of the total cells) produce IL-5 and IL-13 upon PMA and Ionomycin (Cell Stimulation Cocktail) stimulation in all the four cytokine combinations (FIG. 8C). Therefore, IL-2 is a key factor to support ILC2 cell expansion.

### Materials and Methods

ILC2 Cell Isolation

For all experiments, non-mobilized peripheral blood (PB) leukapheresis products were purchased from Memorial Blood Center (St. Paul, Minn.). Human ILC2 cells were enriched by RosetteSep™ human ILC2 enrichment kit (STEMCELL Technologies), or EasySep™ Human ILC2 enrichment kit (STEMCELL Technologies), or the combination of EasySep™ human NK-cell-negative selection kit (STEMCELL Technologies) and EasySep™ human CD56-positive selection II kit (STEMCELL Technologies), per the manufacturer's instructions. Following enrichment, ILC2 cells were sorted by BD FACSAria II as indicated in some experiments.

Cell Culture and Cytokines

ILC2 populations were cultured in either alpha Minimum Essential Media (α-MEM)/20% fetal bovine serum (FBS), or Iscove modified Dulbecco media/10% FBS, both with 1× penicillin/Streptomycin with various cytokine combination in a 5% CO2 incubator at 37° C. Recombinant Human IL-2 (TECIN; Teceleukin) was provided by National Cancer Institute (Frederick, Md.). Recombinant Human IL-17E (IL-25), IL-33 and TSLP were purchased from PeproTech or R&D systems.

Flow Cytometry and Antibodies

Single-cell suspensions were stained for 20 min on ice with the indicated antibodies and Fixable Viability Dye (eBioscience). Human-specific antibodies to the following were purchased from Biolegend: CD1a (HI149), CD11c (3.9), CD14 (HCD14), CD19 (HIB19), CD34 (581), CD45 (2D1), CD94 (DX22), CD123 (6H6), FceR1α (AER-37), TCRαβ (IP26), TCRγδ (B1), CD127 (A019D5), CD161 (HP-3G10), CD294/CRTH2 (BM16), IL-4 (MP4-25D2), IL-5 (JES1-39D10), and IL-13 (JES10-5A2). Human-specific antibodies to CD56 (TULY56), CD303/BDCA2 (AC144), and IFNγ (B27) were purchased from eBioscience, Miltenyi Biotec or BD Biosciences. For intracellular staining, cells were stimulated for 4 hours with 1× Cell Stimulation Cocktail plus protein transport inhibitors (eBioscience) and stained using the BD Fixation/Permeabilization solution kit per manufacturer's instructions. Data were collected using an LSR II instrument (BD Biosciences) and analyzed using FlowJo (Tree Star).

Statistical Analysis

s.d. and P values were determined using Prism software (GraphPad Software, Inc.). P values were calculated using a two-tailed unpaired Student's t-test with 95% confidence interval.

What is claimed is:

1. A method of growing Type 2 innate lymphoid (ILC2) cells, the method comprising:
culturing the ILC2 cells in culture media, the culture media comprising interleukin (IL-2), interleukin 25 (IL-25) and interleukin 33 (IL-33).

2. The method of claim 1 further comprises enriching for ILC2.

3. The method of claim 1, wherein culturing is for about 3 to about 30 days.

4. The method of claim 1, wherein the IL-25 and IL-33 each comprises a concentration of about 30 ng/mL to 100 ng/mL and the IL-2 comprises 10 IU/mL to 50 IU/mL.

5. The method of claim 1, wherein the, IL-25 and IL-33 each comprises a concentration of about 50 ng/mL and the IL-2 comprises 20 IU/mL.

6. The method of claim 1, wherein the culture media further comprises basal media.

7. The method of claim 1, wherein the culture media is free or substantially free of thymic stromal lymphopoietin (TSLP).

8. The method of claim 1, wherein the culture media is free or substantially free of OP9-DL1.

9. The method of claim 1, wherein of culturing ILC2 cells are from blood.

10. The method of claim 1, wherein the percentage of ILC2 cells range from 0.5 to 15%, compared to the initially obtained ILC2 cells of total cells.

11. The method of claim 1, wherein the cells comprises a yield from 1.0×10⁴ to 5.0×10⁶.

12. The method of claim 1, wherein the ILC2 cells comprises an enrichment greater than 10 to 100 fold.

13. The method of claim 1, wherein the method results in an increase in the ILC2 cells by 100 fold relative to enriching ILC2 using EasySep™ ILC2 kit.

14. The method of claim 1, wherein the method results in an increase in the ILC2 cells by 10 fold relative enriching ILC2 using MAC beads.

15. The method of claim 1, wherein the ILC2 cells are from 90% to 100% pure.

16. The method of claim 1, further comprising analyzing the ILC2 for expression of CD161 and GATA3.

17. The method of claim 1, further comprising analyzing the ILC2 for production of interleukin IL-5 and IL-13.

18. A composition comprising ILC2 produced by the method of claim 1.

19. A cell culture media comprising ILC2 cells, IL-2, IL-25, IL-33, and free from or substantially free from OP9-DL, or free from or substantially free from TSLP, or free from or substantially free from IL-7.

20. The cell culture media of claim 19, wherein the IL-25 and IL-33 each comprises a concentration of about 30 ng/ml to 100 ng/ml and the IL-2 comprises 10 IU/mL to 50 IU/mL.
